(19) United States
(12) Patent Application Publication
(10) Pub. No.: US 2025/0043246 A1
(43) Pub. Date: Feb. 6, 2025

(54) ENRICHMENT OF NKX6.1 AND C-PEPTIDE CO-EXPRESSING CELLS DERIVED IN VITRO FROM STEM CELLS

(71) Applicant: Novo Nordisk A/S, Bagsvaerd (DK)

(72) Inventor: Jeannette Schlichting Kirkegaard, Birkeroed (DK)

(21) Appl. No.: 18/926,918

(22) Filed: Oct. 25, 2024

Related U.S. Application Data
(63) Continuation of application No. 16/645,840, filed on Mar. 10, 2020, now abandoned.

(30) Foreign Application Priority Data
Sep. 11, 2017 (EP) 17190412.1

(51) Int. Cl. C12N 5/071; A01N 1/02; A61K 35/39

(52) U.S. Cl. CPC C12N 5/0676; A01N 1/0284; A61K 35/39; C12N 2506/02; C12N 2509/00; C12N 2523/00

(57) ABSTRACT

The present invention relates to method of enriching NKX6.1 and C-peptide co-expressing cell aggregates derived in vitro from stem cells said method comprising the steps of dissociating the endocrine cell aggregates into single cells, treating the single cells with cryopreservation medium and lowering temperature to obtain cryopreserved cells, thawing the cryopreserved cells; and re-aggregating the cells obtained after thawing into endocrine cells.

### CROSS-REFERENCE TO RELATED APPLICATIONS

This application is a continuation of U.S. application Ser. No. 16/645,840, filed Mar. 10, 2020, which is a 35 U.S.C. § 371 National Stage application of International Application PCT/EP2018/074390 (WO 2019/048690), filed Sep. 11, 2018, which claims priority under 35 U.S.C. § 119 to EP Application Serial No. 17190412.1, filed Sep. 11, 2017; the contents of which are incorporated herein by reference.

### TECHNICAL FIELD

The present invention relates to methods of enriching and cryopreserving endocrine cells, NKX2.2 and NKX6.1 or NKX6.1 and C-peptide expressing cells that have been derived in vitro from stem cells.

### BACKGROUND OF THE INVENTION

Although insulin therapy is life-saving, it can be difficult to obtain stable glycemia with exogenous insulin and poor control is associated with serious late state complications (Nathan, D. M., 2014. The diabetes control and complications trial/epidemiology of diabetes interventions and complications study at 30 years: overview. Diabetes care, 37 (1), pp. 9-16). Transplantation of pancreatic islets isolated from human donors to patient with Type1 diabetes have shown good result with some patients becoming completely insulin independent (Barton F. B. et al., 2012. Improvement in Outcomes of Clinical Islet Transplantation: 1999-2010. Diabetes Care, 35 (7), pp. 1436-1445). Despite such advances, one of the major challenges for islet transplantation is limited availability of donor islets. This donor material shortage can be overcome by generating functional insulin secreting cells in vitro by differentiation of human embryonic stem cells. Protocols for generation of functional insulin secreting cells in vitro from stem cells are continuously developing (Pagliuca F. W. et al., 2014. Generation of Functional Human Pancreatic β Cells In vitro. Cell, 159 (2), pp. 428-439; Rezania A et al. Reversal of diabetes with insulin-producing cells derived in vitro from human pluripotent stem cells. Nature Biotechnology, 32 (11), pp. 1121-1133); WO2012175633; WO2014033322; WO2015028614).

Although these protocols are impressive, they generate multiple cells populations, and the ratio among these populations varies from batch-to-batch. A large-scale method for cryopreserving the cells enables quality controls studies to be performed on each cell-batch prior to transplantation and further simplify transplantation logistics. In addition, any method enriching the endocrine populations in the final product is thought to improve transplantation efficacy and safety.

Therefore there is a need for a large scale method for enriching and preserving endocrine populations obtained in vitro by stem cells that not only allows an improvement of phenotype and function but also allows storage and maintenance of cells while batch release studies are performed before transplanting these cells in a subject.

The inventors have found that the method comprising the steps of dissociating, cryopreserving and re-aggregating endocrine cells co-expressing NKX6.1 and C-peptide, or endocrine progenitor cells co-expressing NKX2.2 and NKX6.1, allows to:
enrich cell aggregates with endocrine cells co-expressing NKX6.1 and C-peptide;
reduce the non-endocrine cells (i.e. NKX6.1/C-pep/Glu negative cells) in the cell aggregates;
reduce cluster heterogeneity and cluster size, which reduces variation in vivo;
reduce and control batch-to-batch variation;
store and maintain endocrine and endocrine progenitor cells;
separate the steps of cell production from transplantation, allowing batch tests to be performed.

### SUMMARY OF THE INVENTION

The present invention provides large scale methods for enriching NKX6.1 and C-peptide co-expressing cell aggregates derived in vitro from stem cells. The present method allows enriching cell aggregates derived in vitro from stem cells with endocrine cells co-expressing NKX6.1 and C-peptide or co-expressing NKX2.2 and NKX6.1.

The present invention provides methods for cryopreserving pancreatic endocrine progenitor cells derived in vitro from stem cells comprising the steps of (i) dissociating the cell aggregates into single cells; and (ii) cryopreserving the single cells. The present invention is directed to methods to cryopreserved single endocrine progenitor cells co-expressing NKX2.2 and NKX6.1 or single endocrine cells co-expressing NKX6.1 and C-peptide derived in vitro from stem cells.

The present invention further relates to medical use of the cryopreserved endocrine cells co-expressing NKX6.1 and C-peptide and/or endocrine progenitors cells co-expressing NKX2.2 and NKX6.1 and post cryopreservation inter alia in the treatment of type I diabetes.

The present invention further relates to thawing and re-aggregating the cryopreserved cells into cell aggregates enriched with NKX6.1 and C-peptide co-expressing cells.

The present invention further relates to medical use of re-aggregated endocrine cells co-expressing NKX6.1 and C-peptide or endocrine progenitors cells co-expressing NKX2.2 and NKX6.1 inter alia in the treatment of type I diabetes.

The present invention provides methods for enriching cell aggregates derived in vitro from stem cells with NKX6.1 and C-peptide co-expressing cells while reducing heterogeneity, cluster size and batch to batch variation.

The invention may also solve further problems that will be apparent from the disclosure of the exemplary embodiments.

### BRIEF DESCRIPTION OF DRAWINGS

FIG. 1: Process Overview: Enrichment of NKX6.1 and C-peptide co-expressing cell aggregates

Human embryonic stem cells (hESC) are differentiated in vitro, into endocrine progenitor cells co-expressing NKX2.2 and NKX6.1 or endocrine cells co-expressing NKX6.1 and C-peptide using published protocols (WO2015/028614 and WO2017/144695 respectively). At either stage the cells aggregates are dissociated using enzymatic or non-enzymatic digestion. After dissociation, cells are cryopreserved for example by submerging cells in cryopreservation medium and slowly lowering temperature to −80° C., to obtain cryopreserved cells. These cryopreserved cells are quickly thawed and re-aggregated into cells co-expressing NKX6.1 and C-peptide.

FIG. 2: Dissociation, cryopreservation and re-aggregation of endocrine progenitor cells co-expressing NKX2.2 and NKX6.1: Effects on endocrine phenotype in vitro

A) Enrichment of endocrine cells after cryopreservation, thawing and re-aggregation at the endocrine progenitor stage.

hESC were differentiated into beta like cells and analysed for the distribution of endocrine and non-endocrine cell populations. For each experiment, cells from the same batch were either differentiated using a protocol without (controls) or with a dissociation, cryopreservation and re-aggregation step.

B) Upper panel: endocrine population is measured by the presence of NKX6.1 and C-peptide co-expression using flow cytometry. Results are presented as % change compared to controls.

Lower panel: de-enrichment of non-endocrine cells are shown by a transcriptional decrease in the non-endocrine markers: AFP, GHRL, KRT18 and KRT 8 when cells were generated using a protocol with a dissociation, cryopreservation and re-aggregation steps. Enrichment of functional endocrine cells are characterised by a transcriptional increase in endocrine markers: GIPR, GLP1R and IAPP when cells were generated using a protocol with a dissociation, cryopreservation and re-aggregation step.

C) Decrease in size and heterogeneity after thawing and re-aggregation Upper left panel shows endocrine cell aggregates generated using a protocol without a dissociation, cryopreservation and re-aggregation step.

Lower left panel shows endocrine cell aggregates generated with a dissociation, cryopreservation and re-aggregation step.

Upper right and lower right panels (bar-diagrams) show the cluster size distribution measured using a Biorep® islet counter.

FIG. 3: Dissociation, cryopreservation and re-aggregation of endocrine progenitors cells co-expressing NKX2.2 and NKX6.1: Functionality in vivo

A) Endocrine cells generated after cryopreservation at the endocrine progenitor stage secrete C-peptide when challenged after transplantation into non-diabetic mice

hESC from the same batch were either differentiated without (controls) or with a dissociation, cryopreservation and re-aggregation steps and transplanted under the kidney capsule of non-diabetic mice, or not transplanted (control). To induce C-peptide secretion from the grafts acute insulin resistance was induced by insulin receptor antagonist S961 two weeks after transplantation or by an oral glucose tolerance test seven weeks after transplantation. Human C-peptide was measured 60 and 120 minutes or 20 and 60 minutes after challenge. Cluster formed using a protocol with dissociation, cryopreservation and re-aggregation steps secreted higher levels of C-peptide than those generated using a protocol without a dissociation, cryopreservation and re-aggregation step. Data are presented as mean+/−SEM.

B) Enrichment of NKX6.1 and C-peptide expressing cell aggregates reduce variation in vivo.

The fold increase in C-peptide during the S961 challenge was plotted for animals receiving cells generated using a protocol with or without a dissociation, cryopreservation and re-aggregation step. Efficacy of C-peptide expression was improved using the protocol with dissociation, cryopreservation and re-aggregation and the variation between the animals was reduced.

C) Enrichment of NKX6.1 and C-peptide co-expressing cell aggregates eliminate non-endocrine

cells 8 weeks post transplantation and lead to more homogeneous graft in vivo. 8 weeks post transplantation the mice were terminated and kidneys with grafts were harvested and analysed by immunocytochemistry. Cells were stained for C-peptide, NKX6.1 and glucagon. As indicated by white arrows, areas of non-endocrine cells (NKX6.1-/Glucagon-/C-peptide-) were present in control grafts containing cells generated without a dissociation, cryopreservation and re-aggregation step (4 out of 4 grafts). This was not observed for graft with cells generated using a protocol with a dissociation, cryopreservation and re-aggregation step (0 out of 4 grafts).

FIG. 4: Dissociation, cryopreservation and re-aggregation of endocrine cells co-expressing NKX6.1 and C-peptide: Effects on endocrine cells phenotype in vitro

A) Enrichment of endocrine cells co-expressing NKX6.1 and C-peptide after dissociation, cryopreservation, thawing and re-aggregation at the endocrine cell stage (i.e. BC03).

hESC were differentiated into beta like cells and analysed for the distribution of endocrine and non-endocrine cell populations. For each experiment, cells from the same batch were either differentiated using a protocol without (controls) or with a dissociation, cryopreservation and re-aggregation step.

Right panel: endocrine cell population is measured by the presence of NKX6.1 and C-peptide co-expression using flow cytometry. Results are presented as % change compared to controls.

Left panel: de-enrichment of non-endocrine cells are shown by a transcriptional decrease in the non-endocrine markers: AFP, GHRL, KRT18 and KRT 8 when cells were generated using a protocol with a dissociation, cryopreservation and re-aggregation steps.

B) Decrease in size and heterogeneity after thawing and re-aggregation

Upper left panel shows endocrine cell aggregates generated using a protocol without a dissociation, cryopreservation and re-aggregation step.

Lower left panel shows endocrine cell aggregates generated using a protocol with a dissociation, cryopreservation and re-aggregation step.

Upper right and lower right (bar-diagrams) show the cluster size distribution measured using a Biorep® islet counter.

FIG. 5: Dissociation, cryopreservation and re-aggregation of NKX6.1 and C-peptide co-expressing endocrine cell aggregates: Functionality in vivo

A) Cells dissociated, cryopreserved and re-aggregated at the endocrine cell stage (NKX6.1 and C-peptide co-expressing cells (BC03)) lower blood glucose after transplantation into diabetic Scid-beige mice

hESC were differentiated with a dissociation, cryopreservation and re-aggregation steps and transplanted under the kidney capsule of diabetic mice. After transplantation a fast lowering of blood-glucose is observed.

B) Cells dissociated, cryopreserved and re-aggregated at the endocrine cell stage (NKX6.1 and C-peptide co-expressing cells (BC03)) secrete C-peptide after transplantation into diabetic mice

Basal human C-peptide secretion 20 days after transplantation show that the lowering of blood glucose correlates with human C-peptide secretion.

C) Enrichment of NKX6.1 and C-peptide expressing cell aggregates reduce non-endocrine cells 10 weeks post transplantation.

10 weeks post transplantation the mice were terminated and kidneys with grafts were harvested and analysed by immunocytochemistry. Cells were stained for C-peptide, NKX6.1 and glucagon. As indicated by white arrows, areas of non-endocrine cells (NKX6.1-/Glucagon-/C-peptide-) were present in control grafts containing cells generated without a dissociation, cryopreservation and re-aggregation step (9 out of 11 grafts). This was not observed for graft with cell generated using a protocol with a dissociation, cryopreservation and re-aggregation step (1 out of 3 grafts).

FIG. 6. Dissociation, cryopreservation and re-aggregation of endocrine cells just prior to and early after expression of C-peptide: Effect on glucose responsiveness.

A) Overview of tested differentiation time-points before and after C-peptide expression and effect on glucose responsiveness.

Dissociation, cryopreservation and re-aggregation of cells cryopreserved at different time-points during cell differentiation. Cells were cryopreserved at Pancreatic endoderm stage (PE), 1 day before initiation of C-peptide expression (BC00), 2 days after initiation of C-peptide expression (BC03), 5 days after initiation of C-peptide expression (BC06) and 8 days after initiation of C-peptide expression (BC09) and were all from the same batch of cells. Cells were thawed and differentiated and tested for functionality at 13 days after initiation of C-peptide expression (BC14) in the same setup.

B) Enrichment of NKX6.1 and C-peptide cells by dissociation, cryopreservation and re-aggregation of cells cryopreserved at BC00, BC03, BC06 and BC09, which are in the timeframe about 1 day prior to and about 1 to 8 days after initiation of C-peptide expression.

Expression of NKX6.1 and C-peptide was measured at BC14 using flow cytometry. Data is expressed at % compared to cells from the same batch using a protocol without a dissociation, cryopreservation and re-aggregation step. Results show that enrichment of NKX6.1 and C-peptide cells is the most efficient for cells cryopreserved at BC00 and BC03.

C) Dynamic glucose response when cells are cryopreserved at BC00, BC03, BC06 and BC09.

At the end of the experiment functionality was tested using a dynamic perfusion system. All cells responded to a challenge with 20 mM glucose and exendin-4, but the highest response was observed when cells were cryopreserved at BC00 and BC03, respectively about 1 day prior to and 2 days after initiation of C-peptide expression.

### DETAILED DESCRIPTION

In the broadest sense the present invention relates to methods of enriching and cryopreserving pancreatic endocrine cells derived in vitro from stem cells.

The present invention relates to method for enriching pancreatic cell aggregates with NKX6.1 and NKX2.2 or NKX6.1 and C-peptide co-expressing endocrine cells derived in vitro from stem cells, i.e. embryonic stem cells, or human embryonic stem cells.

The present invention relates to method for enriching cell aggregates with endocrine cells after dissociation, cryopreservation and re-aggregation of endocrine progenitor cells co-expressing NKX6.1 and NKX2.2 or endocrine cells co-expressing NKX6.1 and C-peptide obtained in vitro from stem cells.

The present invention further relates to enriching endocrine progenitor cells and glucose responsive insulin secreting cells derived in vitro from stem cells.

In one aspect, it is described herein a method for selection of endocrine cells from a cell population containing endocrine and non-endocrine cells.

In further aspect, the present method allows to separate the endocrine cells production from the transplantation. For example this allows to transport the cells or to execute quality and safety studies to control batch-to-batch variation before transplantation. In particular, cryopreserved pancreatic endocrine cells obtained according to the method described herein can be store between the steps of production and transplantation, allowing to collect and thaw samples for running purity test(s) (e.g. by flow cytometry) and/or functionality test(s) (e.g. by perfusion of static GSIS).

In further aspect, the present methods allow to obtain homogeneous cryopreserved or re-aggregated endocrine cells co-expressing NKX6.1 and C-peptide or endocrine progenitor cells co-expressing NKX2.2 and NKX6.1 for use in transplantation in a human subject, and for use in treating diabetes.

In one aspect, it is described a method for cryopreserving pancreatic endocrine cell aggregates derived in vitro from stem cells comprising the following steps:
(i) dissociating said endocrine cell aggregates into single cells;
(ii) treating said single cells with cryopreservation medium and lowering temperature, e.g. to at least −80° C., to obtain cryopreserved single cells.

In further aspect the present invention relates to a method of enriching NKX6.1 and C-peptide co-expressing cell aggregates derived in vitro from stem cells said method comprising following steps:
(i) dissociating the cell aggregates into single cells;
(ii) treating the single cells with cryopreservation medium and lowering temperature, e.g. to −80° C., to obtain cryopreserved single cells;
(iii) thawing the cryopreserved cells; and
(iv) cells obtained after thawing and re-aggregation are enriched for NKX6.1 and C-peptide expressing cells.

In one aspect, the present method relates to a method of enriching endocrine cell aggregates derived in vitro from stem cells with endocrine cells co-expressing NKX6.1 and C-peptide or endocrine progenitor cells aggregates co-expressing NKX2.2 and NKX6.1 said method comprising the following steps:
(i) dissociating said endocrine cell aggregates into single cells;
(ii) cryopreserving said single cells, by treating said single cells with cryopreservation medium and lowering temperature, e.g. to at least −80° C., to obtain cryopreserved single cells,
(iii) thawing said cryopreserved endocrine cells; and
(iv) re-aggregating said endocrine cells obtained after thawing.

In a particular embodiment, said endocrine cells of step (i) of the methods described herein are endocrine cells co-expressing NKX6.1 and C-peptide or endocrine progenitor cells co-expressing NKX2.2 and NKX6.1. In a preferred embodiment, said endocrine cells co-expressing NKX6.1 and C-peptide, are endocrine cells wherein C-peptide expression was initiated for up to 7 days, for up to 6 days, for up to 5 days, for up to 4 days, for up to 3 days or for up to 2 days, preferentially for up to 2 days.

In a preferred embodiment, when endocrine cells of step (i) are endocrine progenitor cells aggregates co-expressing NKX2.2 and NKX6.1, said method further comprises a step (v) of differentiating said endocrine progenitor cells co-expressing NKX2.2 and NKX6.1 into endocrine cell aggregates co-expressing NKX6.1 and C-peptide.

In particular, the dissociation step (1) allow to enrich cell aggregates with endocrine cells, as single non-endocrine cells appeared to be less resistant to cryopreservation. Further, the present methods allow reducing variation in in vivo performance, by reducing cluster heterogeneity and cluster size.

Another object of the present invention is the re-aggregated endocrine cells (i.e. cell aggregates obtained following dissociation, cryopreservation and re-aggregation) comprising at least 50%, at least 60%, at least 70%, at least 80%, or at least 90% of endocrine cells co-expressing NKX6.1 and C-peptide.

The cell populations of the re-aggregated cells can be detected and measured with technics known from the person skilled in the art by detecting the markers NKX2.2, NKX6.1 and C-peptide using technic such as FACS.

As used herein, “endocrine cells” or “pancreatic endocrine cells” refers herein to “NKX6.1 and C-peptide co-expressing cells” or “NKX2.2 and NKX6.1 co-expressing cells”, or to endocrine cells selected from 3 days prior to and up to 7 days after the initiation of the expression of C-peptide. Advantageously, endocrine cells described herein are taken from 2 days prior to and up to 5 days after the initiation of the expression of C-peptide, or from 1 day prior to and up to 2 days after the initiation of the expression of C-peptide.

As used herein “NKX6.1 and C-peptide co-expressing cells or cell aggregates” refers to glucose responsive insulin secreting endocrine cells, or to endocrine cells having initiated expression of C-peptide for up to 7 days, up to 6 days, up to 5 days, up to 4 days, up to 3 days or up to 2 days, preferentially for up to 2 days.

“Glucose-responsive insulin secreting cells” or “cells co-expressing NKX6.1 and C-peptide” refers to cells that reside within small cell clusters or cell aggregates called islets of Langerhans in the pancreas. Beta-cells respond to high blood glucose levels by secreting the peptide hormone insulin, which acts on other tissues to promote glucose uptake from the blood, for example in the liver where it promotes energy storage by glycogen synthesis. As used herein “cell aggregate” refers to islet-like cell aggregate obtained after dissociation, cryopreservation and re-aggregation of endocrine cells. As used herein “NKX2.2 and NKX6.1 co-expressing cells” refers to endocrine progenitor cells co-expressing NKX2.2 and NKX6.1, but do not express C-peptide or insulin. Advantageously, “NKX2.2 and NKX6.1 co-expressing cells” refers to cells taken up to 3 days prior C-peptide expression, preferentially up to 2 days prior C-peptide expression, more preferentially up to 1 day prior C-peptide expression.

As used herein “NKX6.1 and C-peptide co-expressing cells or cell aggregates” refers to glucose responsive insulin secreting endocrine cells, or to endocrine cells having initiated expression of C-peptide for up to 7 days, up to 6 days, up to 5 days, up to 4 days, up to 3 days or up to 2 days, preferentially for up to 2 days.

In one aspect, the cell population comprising cell co-expressing NKX6.1 and C-peptide or NKX2.2 and NKX6.1 is obtained from a somatic cell population.

In another aspect, the somatic cell population has been induced to de-differentiate into an embryonic-like stem (ES, e.g., a pluripotent) cell. Such de-differentiated cells are also termed induced pluripotent stem cells (iPSC).

In one embodiment, cell aggregates are dissociated by enzymes or non-enzymatic reagents.

As used herein “enzyme” refers to enzyme suitable for dissociating endocrine cells aggregates derived in vitro from stem cells.

In a preferred embodiment, enzymes or enzyme mixture are selected from a group consisting of protease, protease mixtures, trypsin, collagenase and elastase or mixtures thereof. Preferentially, the enzyme of the present method is selected from enzyme mixture; preferentially the enzyme mixture is Accutase.

In a preferred embodiment, cell aggregates are dissociated by non-enzymatic reagents such as Ethylenediaminetetraacetic acid (EDTA) or ethylene glycol-bis(β-aminoethyl ether)-N,N,N′,N′-tetraacetic acid (EGTA), preferentially the non-enzymatic reagents is EDTA.

In further aspect, the cell population comprising endocrine cells co-expressing NKX6.1 and C-peptide or NKX2.2 and NKX6.1 is obtained from embryonic stem (ES, e.g. pluripotent) cells. In some aspects the cell population comprising endocrine cells co-expressing NKX6.1 and C-peptide or NKX2.2 and NKX6.1 is pluripotent cells such as ES like-cells.

In further aspect, the cell population comprising NKX6.1 and C-peptide or NKX2.2 and NKX6.1 is differentiated from embryonic stem (ES or pluripotent) cells, preferentially from human embryonic stem cells.

In further aspect, the cell population is a population of stem cells. In some aspects the cell population is a population of stem cells differentiated to the endocrine progenitor lineage. In some aspects the cell population is a population of stem cells differentiated to the glucose responsive insulin secreting cells.

Differentiation of protocols of differentiating stem cells into endocrine progenitor cells and glucose-responsive insulin secreting cells are known in the art (WO2015/028614 and WO/2017/144695 respectively).

One object of the present invention is cryopreserved single cells co-expressing NKX2.2 and NKX6.1 or co-expressing NKX6.1 and C-peptide, obtained from dissociating endocrine cell aggregates. In further aspect, the present invention relates to cryopreserved pancreatic endocrine cells obtained according to the method comprising the steps of:
(i) dissociating pancreatic endocrine cell aggregates into single cells;
(ii) treating said single cells with cryopreservation medium and lowering temperature, e.g. at least −80° C., to obtain cryopreserved single cells.

As used herein “lowering temperature to obtain cryopreserved endocrine cells” refers to a step of cooling cells to very low temperatures for a certain period of time, i.e. between −70° C. to −196° C., preferentially to at least −80° C., to prevent any enzymatic or chemical activity which might cause damage to the endocrine single cells of interest.

In one embodiment, the temperature of step (ii) is comprised between −70° C. to −196° C., between −80° C. to −160° C., or between −80° C. to −120° C., preferentially the temperature of step (ii) is at least −80° C. In one embodiment, the temperature is lowered in one step or in step-wise to obtain cryopreserved cells, preferentially the temperature is lowered in one step.

As used herein “cryopreserved cells” or “cryopreserved single cells” refers to cells that have been obtained after cell aggregates have been dissociated into single cells, treated with a cryopreservation medium and cryopreserved by lowering temperature to very low temperature, e.g. between −70° C. to −196° C.

As used herein “cryopreservation medium” refers to medium which is suitable to maintain integrity of the endocrine cells or endocrine progenitor cells during the cryopreservation step. Most cryopreservation media contain DMSO, serum or synthetic serum substitutes, and are buffered for pH using for example HEPES of sodiumbicarbonate.

In one embodiment, cryopreservation medium comprises compounds selected from Dimethyl sulfoxide (DMSO), serum, synthetic serum substitutes or glycerol.

In accordance with the present invention, cryopreserved cells described herein can be stored for at least one hour, at least one day, at least one week, at least one month, at least two months, at least three months, at least one year or any time period between any times provided in this range.

In one embodiment cryopreserved cells or re-aggregated endocrine cells described herein may be used in the treatment of diabetes, e.g. by implantation into a patient in need of such treatment.

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation.

Stem cells are classified by their developmental potential as: (1) totipotent, meaning able to give rise to all embryonic and extraembryonic cell types; (2) pluripotent, meaning able to give rise to all embryonic cell types; (3) multi-potent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multi-potent stem cells; and (5) unipotent, meaning able to give rise to a single cell lineage (e.g., spermatogenic stem cells).

As used herein “differentiate” or “differentiation” refers to a process where cells progress from an undifferentiated state to a differentiated state, from an immature state to a less immature state or from an immature state to a mature state. For example, early undifferentiated embryonic pancreatic cells are able to proliferate and express characteristics markers, like PDX1, NKX6.1, and PTF1a. Mature or differentiated pancreatic cells do not proliferate and do secrete high levels of pancreatic endocrine hormones or digestive enzymes. e.g., fully differentiated beta cells secrete insulin at high levels in response to glucose. Changes in cell interaction and maturation occur as cells lose markers of undifferentiated cells or gain markers of differentiated cells. Loss or gain of a single marker can indicate that a cell has “matured or fully differentiated”. The term “differentiation factor” refers to a compound added to pancreatic cells to enhance their differentiation to mature endocrine cells also containing insulin producing beta cells. Exemplary differentiation factors include hepatocyte growth factor, keratinocyte growth factor, exendin-4, basic fibroblast growth factor, insulin-like growth factor-1, nerve growth factor, epidermal growth factor platelet-derived growth factor, and glucagon-like peptide 1. In some aspects differentiation of the cells comprises culturing the cells in a medium comprising one or more differentiation factors.

Mature or differentiated pancreatic cells do not proliferate and do secrete high levels of pancreatic endocrine hormones or digestive enzymes, e.g., fully differentiated beta-cells secrete insulin at high levels in response to glucose. Changes in cell interaction and maturation occur as cells lose markers of undifferentiated cells or gain markers of differentiated cells. Loss or gain of a single marker can indicate that a cell has “matured or fully differentiated”.

The term “differentiation factor” refers to a compound added to ES- or pancreatic precursor cells to enhance their differentiation to EP cells. Differentiation factors may also drive further differentiation into mature beta-cells.

Exemplary differentiation factors include hepatocyte growth factor, keratinocyte growth factor, exendin-4, basic fibroblast growth factor, insulin-like growth factor-1, nerve growth factor, epidermal growth factor platelet-derived growth factor, glucagon-like peptide 1, indolactam V, IDE1&2 and retinoic acid.

In some aspects differentiation of the cells comprises culturing the cells in a medium comprising one or more differentiation factors.

In one embodiment the invention relates to a method of providing pancreatic endocrine function to a mammal deficient in its production of at least one pancreatic hormone, the method comprising the steps of implanting endocrine cells obtained by any of the methods of the invention in an amount sufficient to produce a measurable amount of said at least one pancreatic hormone in said mammal.

As used herein, the term “human pluripotent stem (hPS) cells” refers to cells that may be derived from any source and that are capable, under appropriate conditions, of producing human progeny of different cell types that are derivatives of all of the 3 germinal layers (endoderm, mesoderm, and ectoderm). hPS cells have the ability to form a teratoma in 8-12 week old SCID mice and/or the ability to form identifiable cells of all three germ layers in tissue culture. Included in the definition of human pluripotent stem cells are embryonic cells of various types including human blastocyst derived stem (hBS) cells in the literature often denoted as human embryonic stem (hES) cells.

In one aspect, it is described herein a method for cryopreserving endocrine cell aggregates derived in vitro from stem cells comprising the following steps:
(i) dissociating said endocrine cell aggregates into single cells;
(ii) cryopreserving said single cells, wherein the endocrine cells are endocrine progenitor cells co-expressing NKX6.1 and NKX2.2 or endocrine cells co-expressing NKX6.1 and C-peptide, wherein C-peptide expression was initiated for up to 7 days, up to 6 days, up to 5 days, up to 4 days, up to 3 days or up to 2 days, preferentially for up to 2 days.

In another aspect, it is described herein a method of enriching cell aggregates with endocrine cell co-expressing NKX6.1 and C-peptide or endocrine progenitor cells co-expressing NKX2.2 and NKX6.1 derived in vitro from stem cells said method comprising following steps:
(i) dissociating said cell aggregates into single cells;
(ii) cryopreserving said single cells;
(iii) thawing said cryopreserved single cells; and
(iv) cells obtained after thawing and re-aggregation are enriched for NKX6.1 and C-peptide expressing cells;
wherein the endocrine cells are endocrine progenitor cells co-expressing NKX2.2 and NKX6.1 or endocrine cells co-expressing NKX6.1 and C-peptide, wherein endocrine cells C-peptide expression was initiated for up to 7 days, up to 6 days, up to 5 days, up to 4 days, up to 3 days, up to 2 days, preferentially for up to 2 days.

In one aspect, it is described herein re-aggregated endocrine cells co-expressing NKX6.1 and C-peptide or NKX2.2 and NKX6.1 obtained according to the method of the invention.

In one aspect, it is described herein re-aggregated endocrine cells comprising at least 50% of endocrine cells co-expressing NKX6.1 and C-peptide and/or endocrine progenitor cells co-expressing NKX2.2 and NKX6.1.

In one embodiment, it is described herein re-aggregated endocrine cells comprising at least 60% of endocrine cells co-expressing NKX6.1 and C-peptide or endocrine progenitor cells co-expressing NKX2.2 and NKX6.1.

In one embodiment, it is described herein re-aggregated endocrine cells comprising at least 70% of endocrine cells co-expressing NKX6.1 and C-peptide or endocrine progenitor cells co-expressing NKX2.2 and NKX6.1.

In one embodiment, it is described herein re-aggregated endocrine cells comprising at least 80% of endocrine cells co-expressing NKX6.1 and C-peptide or endocrine progenitor cells co-expressing NKX2.2 and NKX6.1.

In one aspect, re-aggregated endocrine cells described herein are used as a medicament.

In one aspect, re-aggregated endocrine cells described herein are used in treating diabetes.

Further, the composition comprising re-aggregated endocrine cells co-expressing NKX6.1 and C-peptide or co-expressing NKX2.2 and NKX6.1 described herein are used in treating diabetes.

In further aspect, it is described herein a medicament comprising cell aggregates enriched with endocrine cells according to the present description. In a preferred embodiment, the medicament described herein comprises re-aggregated endocrine cells co-expressing NKX6.1 and C-peptide and/or co-expressing NKX2.2 and NKX6.1 as described herein.

In further aspect, it is described herein a device comprising cryopreserved endocrine cells, or re-aggregated endocrine cells, or a composition containing re-aggregated endocrine cells, or a cell aggregates, or a medicament as described herein.

The various methods and other embodiments described herein may require or utilise hPS cells from a variety of sources. For example, hPS cells suitable for use may be obtained from developing embryos. Additionally or alternatively, suitable hPS cells may be obtained from established cell lines and/or human induced pluripotent stem (hiPS) cells.

As used herein, the term “hiPS cells” refers to human induced pluripotent stem cells.

As used herein, the term “blastocyst-derived stem cell” is denoted BS cell, and the human form is termed “hBS cells”. In literature such cells are often referred to as embryonic stem cells, and more specifically human embryonic stem cells (hESC). The pluripotent stem cells in turn used in the present invention can thus be embryonic stem cells prepared from blastocysts, as described in e.g. WO 03/055992 and WO 2007/042225, or be commercially available hBS cells or cell lines. However, it is further envisaged that any human pluripotent stem cell in turn can be used in the present invention, including differentiated adult cells which are reprogrammed to pluripotent cells by e.g. the treating adult cells with certain transcription factors, such as OCT4, SOX2, NANOG, and LIN28.

As used herein “viability” of a cell or “viable cell” refers to capability of normal growth and development after having been cryopreserved, thawed and/or re-aggregated. In one aspect at least 10%, 20%, 30%, 40%, 50%, 60%, 70%, 80%, 90% re-aggregated endocrine cells are viable.

The present invention may also solve further problems that will be apparent from the disclosure of the exemplary embodiments.

Unless otherwise indicated in the specification, terms presented in singular form also include the plural situation.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Further Embodiments of the Invention

Embodiment 1: A method for enriching NKX6.1 and C-peptide expressing cell aggregates derived in vitro from stem cells said method comprising following steps:
(i) dissociating the cell aggregates into single cells;
(ii) treating the single cells with cryopreservation medium and lowering temperature, e.g. to −80° C., to obtain cryopreserved cells;
(iii) thawing the cryopreserved cells; and
(iv) cells obtained after thawing and re-aggregation are enriched for NKX6.1 and C-peptide co-expressing cells.

Embodiment 2: The method of embodiment 1, wherein NKX6.1 and C-peptide expressing cell aggregates are endocrine progenitor cells or glucose responsive insulin secreting cells, preferentially said NKX6.1 and C-peptide expressing cell aggregates are endocrine cells that have been expressing C-peptide for up to 7 days, up to 6 days, up to 5 days, up to 4 days, up to 3 days, up to 2 days, preferentially for up to 2 days.

Embodiment 3: The method of embodiment 2, wherein endocrine progenitor cells co-express NKX2.2 and NKX6.1.

Embodiment 4: The method of anyone of embodiments 1 to 3, wherein stem cells are induced pluripotent stem cells.

Embodiment 5: The method of anyone of embodiments 1 to 3, wherein stem cells are embryonic stem cells.

Embodiment 6: The method of anyone of embodiments 1 to 3, wherein stem cells are human embryonic stem cells.

Embodiment 7: The method of anyone of embodiments 1 to 6, wherein NKX6.1 and C-peptide expressing cell aggregates derived in vitro from stem cells that have been differentiated into definitive endoderm.

Embodiment 8: The method of anyone of embodiments 1 to 6, wherein NKX6.1 and C-peptide expressing cell aggregates derived in vitro from stem cells that have been differentiated into pancreatic endoderm.

Embodiment 9: The method of anyone of embodiments 1 to 6, wherein NKX6.1 and C-peptide expressing cell aggregates derived in vitro from stem cells that have been differentiated into endocrine progenitor cells.

Embodiment 10: The method of anyone of embodiments 1 to 6, wherein NKX6.1 and C-peptide expressing cell aggregates derived in vitro from stem cells that have been differentiated into endocrine progenitor cells expressing NKX2.2 and NKX6.1.

Embodiment 11: The method of anyone of embodiments 1 to 6, wherein NKX6.1 and C-peptide expressing cell aggregates derived in vitro from stem cells that have been differentiated into glucose responsive insulin secreting cells.

Embodiment 12: The method of anyone of embodiments 1 to 11, wherein NKX6.1 and C-peptide expressing cell aggregates are dissociated by enzymes.

Embodiment 13: The method of embodiment 12, wherein NKX6.1 and C-peptide expressing cell aggregates are dissociated by enzymes selected from a group consisting of protease or protease mixtures.

Embodiment 14: The method of embodiment 12, wherein NKX6.1 and C-peptide expressing cell aggregates are dissociated by enzymes selected from a group consisting of Trypsin, collagenase and elastase or mixtures thereof.

Embodiment 15: The method of embodiment 12, wherein NKX6.1 and C-peptide expressing cell aggregates are dissociated by Accutase enzyme.

Embodiment 16: The method of embodiment 15, wherein Accutase is a mixture of protease and collagenase.

Embodiment 17: The method of anyone of embodiments 1 to 11, wherein NKX6.1 and C-peptide expressing cell aggregates are dissociated by non-enzymatic reagents.

Embodiment 18: The method of embodiment 17, wherein NKX6.1 and C-peptide expressing cell aggregates are dissociated by non-enzymatic reagents such as Ethylenediaminetetraacetic acid (EDTA) or ethylene glycol-bis(3-aminoethyl ether)-N,N,N′,N′-tetraacetic acid (EGTA).

Embodiment 19: The method of anyone of embodiments 1 to 18, wherein cryopreservation medium is with a cryoprotectant.

Embodiment 20: The method of embodiment 19, wherein the cryoprotectant is Dimethyl sulfoxide (DMSO).

Embodiment 21: The method of anyone of embodiments 1 to 18, wherein cryopreservation medium is without a cryoprotectant.

Embodiment 22: The method of anyone of embodiments 1 to 21, wherein after treatment of single cells with cryopreservation medium the temperature is lowered between −70° C. to −196° C., between-80° C. to −160° C., or between −80° C. to −120° C., or −80° C., in one step to obtain cryopreserved cells.

Embodiment 23: The method of anyone of embodiments 1 to 21, wherein after treatment of single cells with cryopreservation medium the temperature is lowered between −70° C. to −196° C., between-80° C. to −160° C., or between −80° C. to −120° C., or −80° C., step-wise to obtain cryopreserved cells.

Embodiment 24: The method of anyone of embodiments 1 to 21, wherein after treatment of single cells with cryopreservation medium the temperature is lowered to −80° C. in one step to obtain cryopreserved cells.

Embodiment 25: The method of anyone of embodiments 1 to 24, wherein cryopreserved cells obtained by the steps (i) and (ii) of embodiment 1 co-express NKX2.2 and NKX6.1.

Embodiment 26: The method of anyone of embodiments 1 to 24, wherein at least 20% of cryopreserved cells obtained by the steps (i) and (ii) of embodiment 1-express NKX2.2 and NKX6.1.

Embodiment 27: The method of anyone of embodiments 1 to 24, wherein at least 40% of cryopreserved cells obtained by the steps (i) and (ii) of embodiment 1 express NKX2.2 and NKX6.1.

Embodiment 28: The method of anyone of embodiments 1 to 24, wherein at least 60% of cryopreserved cells obtained by the steps (i) and (ii) of embodiment 1 express NKX2.2 and NKX6.1.

Embodiment 29: The method of anyone of embodiments 1 to 24, wherein at least 80% of cryopreserved cells obtained by the steps (i) and (ii) of embodiment 1 express NKX2.2 and NKX6.1.

Embodiment 30: The method of anyone of embodiments 1 to 24, wherein cryopreserved cells co-express NKX6.1 and C-peptide.

Embodiment 31: The method of anyone of embodiments 1 to 24, wherein at least 20% of cryopreserved cells obtained by the steps (i) and (ii) of embodiment 1 express NKX6.1 and C-peptide.

Embodiment 32: The method of anyone of embodiments 1 to 24, wherein at least 40% or 50% of cryopreserved cells obtained by the steps (i) and (ii) of embodiment 1 express NKX6.1 and C-peptide.

Embodiment 33: The method of anyone of embodiments 1 to 24, wherein at least 60% or 70% of cryopreserved cells obtained by the steps (i) and (ii) of embodiment 1 express NKX6.1 and C-peptide.

Embodiment 34: The method of anyone of embodiments 1 to 24, wherein at least 80% of cryopreserved cells obtained by the steps (i) and (ii) of embodiment 1 express NKX6.1 and C-peptide.

Embodiment 35: The method of anyone of embodiments 1 to 34, wherein cryopreserved cells are viable.

Embodiment 36: The method of anyone of embodiments 1 to 34, wherein at least 20% of cryopreserved cells obtained by the steps (i) and (ii) of embodiment 1 are viable.

Embodiment 37: The method of anyone of embodiments 1 to 34, wherein at least 40% of cryopreserved cells obtained by the steps (i) and (ii) of embodiment 1 are viable.

Embodiment 38: The method of anyone of embodiments 1 to 34, wherein at least 60% of cryopreserved cells obtained by the steps (i) and (ii) of embodiment 1 are viable.

Embodiment 39: The method of anyone of embodiments 1 to 34, wherein at least 80% of cryopreserved cells obtained by the steps (i) and (ii) of embodiment 1 are viable.

Embodiment 40: Cryopreserved cells obtained by the steps (i) and (ii) of the method of anyone of embodiments 1 to 39.

Embodiment 41: Cryopreserved cells obtained by the steps (i) and (ii) of the method of anyone of embodiments 1 to 39 are stored for at least 7 days, preferentially for at least 14 days.

Embodiment 42: Cryopreserved cells obtained by the steps (i) and (ii) of the method of anyone of embodiments 1 to 39 are stored for at least 21 days.

Embodiment 43: Cryopreserved cells obtained by the steps (i) and (ii) of the method of anyone of embodiments 1 to 39 are stored for at least 1 month.

Embodiment 44: Cryopreserved cells obtained by the steps (i) and (ii) of the method of anyone of embodiments 1 to 39 are stored for at least 2 months.

Embodiment 45: Cryopreserved cells obtained by the steps (i) and (ii) of the method of anyone of embodiments 1 to 39 are stored for at least 3 months.

Embodiment 46: Cryopreserved cells obtained by the steps (i) and (ii) of the method of anyone of embodiments 1 to 39 are stored for at least 1 year.

Embodiment 47: Cryopreserved cells obtained by the steps (i) and (ii) of the method of anyone of embodiments 1 to 39 for use for further differentiation.

Embodiment 48: Cryopreserved cells obtained by the steps (i) and (ii) of the method of anyone of embodiments 1 to 39 for use for encapsulation.

Embodiment 49: Cryopreserved cells obtained by the steps (i) and (ii) of the method of anyone of embodiments 1 to 39 for use for encapsulation into a device.

Embodiment 50: Cryopreserved cells obtained by the steps (i) and (ii) of the method of anyone of embodiments 1 to 39 for use for transplantation into a subject.

Embodiment 51: Cryopreserved cells obtained by the steps (i) and (ii) of the method of anyone of embodiments 1 to 39 for use for transplantation into a mammal.

Embodiment 52: Cryopreserved cells obtained by the steps (i) and (ii) of the method of anyone of embodiments 1 to 39 for use for transplantation into human.

Embodiment 53: The method according to anyone of embodiments 1 to 39, wherein cryopreserved cells are thawed in the presence of Rock inhibitor.

Embodiment 54: The method according to embodiment 53, wherein cryopreserved cells are thawed in the presence of 10 UM of Rock inhibitor.

Embodiment 55: The method according to anyone of embodiments 1 to 39, wherein cryopreserved cells are thawed in the absence of Rock inhibitor.

Embodiment 56: The method according to anyone of embodiments 1 to 39, and 53 to 55, wherein cells obtained after thawing are re-aggregated.

Embodiment 57: The method according to anyone of embodiments 1 to 39, and 53 to 55, wherein cells obtained after thawing are re-aggregated for at least 2 days.

Embodiment 58: Re-aggregated cells obtained by method according to anyone of embodiments 1 to 39, and 53 to 57.

Embodiment 59: The method according to anyone of embodiments 1 to 39, and 53 to 57, wherein said re-aggregated cells co-express NKX6.1 and C-peptide.

Embodiment 60: The method according to embodiment 59, wherein at least 20% of re-aggregated cells co-express NKX6.1 and C-peptide.

Embodiment 61: The method according to embodiment 59, wherein at least 40% of re-aggregated cells co-express NKX6.1 and C-peptide.

Embodiment 62: The method according embodiment 59, wherein at least 60% of re-aggregated cells co-express NKX6.1 and C-peptide.

Embodiment 63: The method according embodiment 59, wherein at least 80% of re-aggregated cells co-express NKX6.1 and C-peptide.

Embodiment 64: The method according to embodiment 59, wherein at least 20% of re-aggregated cells are glucose responsive insulin secreting cells.

Embodiment 65: The method according to embodiment 59, wherein at least 40% of re-aggregated cells are glucose responsive insulin secreting cells.

Embodiment 66: The method according to embodiment 59, wherein at least 60% of re-aggregated cells are glucose responsive insulin secreting cells.

Embodiment 67: The method according to embodiment 59, wherein at least 80% of re-aggregated cells are glucose responsive insulin secreting cells.

Embodiment 68: Re-aggregated cells obtained by method according to anyone of embodiments 1 to 39, 53 to 57 and 59 to 66, for use for further differentiation.

Embodiment 69: Re-aggregated cells obtained by method according to anyone of embodiments 1 to 39, 53 to 57 and 59 to 66, for use for encapsulation.

Embodiment 70: Re-aggregated cells obtained by method according to anyone of embodiments 1 to 39, 53 to 57 and 59 to 66, for use for encapsulation into a device.

Embodiment 71: Re-aggregated cells obtained by method according to anyone of embodiments 1 to 39, 53 to 57 and 59 to 66, for use for transplantation into a subject.

Embodiment 72: Re-aggregated cells obtained by method according to anyone of embodiments 1 to 39, 53 to 57 and 59 to 66, for use for transplantation into a mammal.

Embodiment 73: Re-aggregated cells obtained by method according to anyone of embodiments 1 to 39, 53 to 57 and 59 to 66, for use for transplantation into human.

Embodiment 74: A method for cryopreserving NKX2.2 and NKX6.1 or NKX6.1 and C-peptide co-expressing cell aggregates derived in vitro from stem cells said method comprising following steps:
(i) dissociating the cell aggregates into single cells;
(ii) treating the single cells with cryopreservation medium and lowering temperature, e.g. to at least −80° C., to obtain cryopreserved cells.

Embodiment 75: The method according to embodiment 74, wherein cryopreserved cells are thawed.

Embodiment 76: The method according to embodiment 75, wherein cryopreserved cells that have been thawed are re-aggregated.

Embodiment 77: The method according to embodiment 76, wherein cryopreserved cells that have been re-aggregated co-express NKX6.1 and C-peptide.

Embodiment 78: The method according to embodiment 74, wherein NKX2.2 and NKX6.1 co-expressing cell aggregates are endocrine progenitor cells.

Embodiment 79: The method according to anyone of embodiments 74 to 78, wherein stem cells are induced pluripotent stem cells.

Embodiment 80: The method according to anyone of embodiments 74 to 78, wherein stem cells are embryonic stem cells.

Embodiment 81: The method according to anyone of embodiments 74 to 78, wherein stem cells are human embryonic stem cells.

Embodiment 82: The method according to anyone of embodiments 74 to 81, wherein NKX2.2 and NKX6.1 co-expressing cell aggregates derived in vitro from stem cells that have been differentiated into definitive endoderm.

Embodiment 83: The method according to anyone of embodiments 74 to 81, wherein NKX2.2 and NKX6.1 co-expressing cell aggregates derived in vitro from stem cells that have been differentiated into pancreatic endoderm, i.e. co-expressing PDX-1/NKX6.1.

Embodiment 84: The method of anyone of embodiments 74 to 81, wherein NKX2.2 and NKX6.1 co-expressing cell aggregates derived in vitro from stem cells that have been differentiated into endocrine progenitors.

Embodiment 85: The method of anyone of embodiments 74 to 84, wherein NKX2.2 and NKX6.1 co-expressing cell aggregates are dissociated by enzymes.

Embodiment 86: The method of embodiment 85, wherein said enzymes are selected from a group consisting of protease or protease mixtures or protease and collagenase mixtures.

Embodiment 87: The method of embodiment 85, wherein said enzymes are selected from a group consisting of Trypsin, collagenase and elastase or mixtures thereof.

Embodiment 88: The method of embodiment 85, wherein said enzymes are Accutase enzyme.

Embodiment 89: The method of embodiment 88, wherein Accutase is a mixture of protease and collagenase.

Embodiment 90: The method of anyone of embodiments 74 to 84, wherein NKX2.2 and NKX6.1 co-expressing cell aggregates are dissociated by non-enzymatic reagents.

Embodiment 91: The method of embodiment 90, wherein said non-enzymatic reagents is selected from Ethylenediaminetetraacetic acid (EDTA) or ethylene glycol-bis(β-aminoethyl ether)-N,N,N′,N′-tetraacetic acid (EGTA).

Embodiment 92: The method of anyone of embodiments 74 to 91, wherein cryopreservation medium is with a cryoprotectant.

Embodiment 93: The method of embodiment 92, wherein the cryoprotectant is Dimethyl sulfoxide (DMSO).

Embodiment 94: The method of anyone of embodiments 74 to 91, wherein cryopreservation medium is without a cryoprotectant.

Embodiment 95: The method of anyone of embodiments 74 to 94, wherein after treatment of single cells with cryopreservation medium the temperature is lowered between −70° C. to −196° C., between −80° C. to −160° C., or between −80° C. to −120° C., or to −80° C., in one step to obtain cryopreserved cells.

Embodiment 96: The method of anyone of embodiments 74 to 94, wherein after treatment of single cells with cryopreservation medium the temperature is lowered between −70° C. to −196° C., between −80° C. to −160° C., or between −80° C. to −120° C., or −80° C., step-wise to obtain cryopreserved cells.

Embodiment 97: Cryopreserved cells obtained by the method according to anyone of embodiments 74 to 96.

Embodiment 98: Cryopreserved cells according to embodiment 97, wherein cryopreserved cells co-express NKX2.2 and NKX6.1.

Embodiment 99: Cryopreserved cells according to embodiment 97, wherein at least 20% of cryopreserved cells co-express NKX2.2 and NKX6.1.

Embodiment 100: Cryopreserved cells according to embodiment 97, wherein at least 40% of cryopreserved cells co-express NKX2.2 and NKX6.1.

Embodiment 101: Cryopreserved cells according to embodiment 97, wherein at least 60% of cryopreserved cells co-express NKX2.2 and NKX6.1.

Embodiment 102: Cryopreserved cells according to embodiment 97, wherein at least 80% of cryopreserved cells co-express NKX2.2 and NKX6.1.

Embodiment 103: Cryopreserved cells obtained according to embodiment 97 can be stored for at least 7 days.

Embodiment 104: Cryopreserved cells obtained according to embodiment 97 can be stored for at least 14 days.

Embodiment 105: Cryopreserved cells obtained according to embodiment 97 can be stored for at least 21 days.

Embodiment 106: Cryopreserved cells obtained according to embodiment 97 can be stored for at least 1 month.

Embodiment 107: Cryopreserved cells obtained according to embodiment 97 can be stored for at least 2 months.

Embodiment 108: Cryopreserved cells obtained according to embodiment 97 can be stored for at least 3 months.

Embodiment 109: Cryopreserved cells obtained according to embodiment 97 can be stored for at least 6 months.

Embodiment 110: Cryopreserved cells obtained according to anyone of embodiment 97-109 for use for further differentiation.

Embodiment 111: Cryopreserved cells obtained according to anyone of embodiment 97-109 for use for encapsulation.

Embodiment 112: Cryopreserved cells obtained according to anyone of embodiments 97 to 109 for use for encapsulation into a device.

Embodiment 113: Cryopreserved cells obtained according to anyone of embodiments 97 to 109 for use for transplantation into a subject.

Embodiment 114: Cryopreserved cells obtained according to anyone of embodiments 97 to 109 for use for transplantation into a mammal.

Embodiment 115: Cryopreserved cells obtained according to anyone of embodiments 97 to 109 for use for transplantation into human.

Embodiment 116: The method according to anyone of embodiments 74 to 96, wherein cryopreserved cells are thawed in the presence of Rock inhibitor.

Embodiment 117: The method of embodiment 116, wherein cryopreserved cells are thawed in the presence of 10 UM of Rock inhibitor.

Embodiment 118: The method according to anyone of embodiments 74 to 96, wherein cryopreserved cells are thawed in the absence of Rock inhibitor.

Embodiment 119: The method according to anyone of embodiments 74 to 96, wherein cells obtained after thawing are re-aggregated.

Embodiment 120: The method according to anyone of embodiments 74 to 96, wherein cells obtained after thawing are re-aggregated for 2 days.

Embodiment 121: Re-aggregated cells obtained by method of according to anyone of embodiments 76 to 96 and 116-120.

Embodiment 122: The method according to anyone of embodiments 76 to 96 and 116 to 120, wherein re-aggregated cells co-express NKX6.1 and C-peptide.

Embodiment 123: The method according to anyone of embodiments 76 to 96 and 116 to 120, wherein at least 20% of re-aggregated cells express NKX6.1 and C-peptide.

Embodiment 124: The method according to anyone of embodiments 76 to 96 and 116 to 120, wherein at least 40% of re-aggregated cells express NKX6.1 and C-peptide.

Embodiment 125: The method according to anyone of embodiments 76 to 96 and 116 to 120, wherein at least 60% of re-aggregated cells express NKX6.1 and C-peptide.

Embodiment 126: The method according to anyone of embodiments 76 to 96 and 116 to 120, wherein at least 80% of re-aggregated cells express NKX6.1 and C-peptide.

Embodiment 127: The method according to anyone of embodiments 76 to 96 and 116 to 120, wherein at least 20% of re-aggregated cells are glucose responsive insulin secreting cells.

Embodiment 128: The method according to anyone of embodiments 76 to 96 and 116 to 120, wherein at least 40% of re-aggregated cells are glucose responsive insulin secreting cells.

Embodiment 129: The method according to anyone of embodiments 76 to 96 and 116 to 120, wherein at least 60% of re-aggregated cells are glucose responsive insulin secreting cells.

Embodiment 130: The method according to anyone of embodiments 76 to 96 and 116 to 120, wherein at least 80% of re-aggregated cells are glucose responsive insulin secreting cells.

Embodiment 131: Re-aggregated cells obtained by method according to anyone of embodiments to anyone of embodiments 76 to 96 and 116 to 120 for use for further differentiation.

Embodiment 132: Re-aggregated cells obtained by method according to anyone of embodiments 76 to 96 and 116 to 120 for use for encapsulation.

Embodiment 133: Re-aggregated cells obtained by method according to anyone of embodiments 76 to 96 and 116 to 120 for use for encapsulation into a device.

Embodiment 134: Re-aggregated cells obtained by method according to anyone of embodiments 76 to 96 and 116 to 120 for use for transplantation into a subject.

Embodiment 135: Re-aggregated cells obtained by method according to anyone of embodiments 76 to 96 and 116 to 120 for use for transplantation into a mammal.

Embodiment 136: Re-aggregated cells obtained by method according to anyone of embodiments 76 to 96 and 116 to 120 for use for transplantation into human.

Embodiment 137: Re-aggregated cells obtained by method according to anyone of embodiments 76 to 96 and 116 to 120 for use as a medicament.

Embodiment 138: Re-aggregated cells obtained by method according to anyone of embodiments 76 to 96 and 116 to 120 for use in treating diabetes.

Embodiment 139: Re-aggregated endocrine cells comprising at least 60%, at least 70%, at least 80%, or at least 90% of endocrine cells co-expressing NKX6.1 and C-peptide.

Embodiment 140: Re-aggregated endocrine cells comprising at least 60%, at least 70%, at least 80%, or at least 90% of endocrine progenitor cells co-expressing NKX2.2 and NKX6.1.

Embodiment 141: Re-aggregated endocrine cells obtained according to the method of enriching endocrine cell aggregates according to anyone of embodiments 1 to 39, 76 to 96 and 116 to 120.

Embodiment 142: Re-aggregated endocrine cells according to anyone of the embodiments 138 to 140 for use as a medicament.

Embodiment 143: Re-aggregated endocrine cells according to anyone of the embodiments 138 to 140 for use in treating diabetes.

Embodiment 144: Process for the preparation of a medicament for treating diabetes using re-aggregated cells according to anyone of embodiments 68 to 73 and 131 to 143.

Embodiment 145: Cryopreserved single endocrine cells co-expressing NKX2.2 and NKX6.1 or single endocrine cells co-expressing NKX6.1 and C-peptide.

Embodiment 146: Cryopreserved single endocrine cells co-expressing NKX2.2 and NKX6.1 or co-expressing NKX6.1 and C-peptide obtained according to the method of cryopreserving according to anyone of the embodiments 74 to 96, and 116 to 120.

Embodiment 147: Cryopreserved single endocrine cells according to anyone of embodiment 145 or 146 for use in the transplantation into a subject.

Embodiment 148: Cryopreserved single endocrine cells according to anyone of embodiment 145 or 146 for use in treating diabetes

Embodiment 149: Cryopreserved single endocrine cells according to anyone of embodiment 145 or 146 for use in the transplantation into a subject.

Embodiment 150: Cryopreserved single endocrine cells according to anyone of embodiment 145 or 146 for use as a medicament.

Embodiment 151: Composition containing re-aggregated cells obtained by method according to anyone of embodiments 1 to 39, 76 to 96, 116 to 120 and 122 to 130 for use as medicament.

Embodiment 152: Composition containing re-aggregated cells obtained by method according to anyone of embodiments 1 to 39, 76 to 96, 116 to 120 and 122 to 130 for use in treating diabetes.

Embodiment 153: Composition containing re-aggregated endocrine cells according to the embodiment 131 to 143 for use as a medicament or for use in treating diabetes, e.g. Type I diabetes.

Embodiment 154: Medicament containing re-aggregated cells obtained by method according to anyone of embodiments 1 to 39, 76 to 96, 116 to 120 and 122 to 130.

Embodiment 155: Medicament comprising re-aggregated endocrine cells according any of the embodiments 131 to 143.

Embodiment 156: A device comprising cryopreserved endocrine cells according to anyone of embodiments 40 to 52, 97 to 115 and 145 to 150, or re-aggregated endocrine cells according to anyone of embodiments 58, 68 to 73, 131 to 143, or 149 to 153, or a composition according to embodiment 151 to 153, or a medicament according to embodiment 154 or 155.

Surprisingly, an enriched population of endocrine cells is obtained by carrying out the process of the present invention. The enriched endocrine cells have a homogeneous and small cluster size that renders them suitable for transplantation into a subject.

### EXAMPLES

### List of Abbreviations

Alk5i II: TGFβ kinase/activin receptor-like kinase
DAPT: Difluorophenylacetyl)-alanyl-phenylglycine-t-butyl-ester
DMBI: (Z)-3-[4-(Dimethylamino)benzylidenyl]indolin-2-one
DZNEP: 3-Deazaneplanocin A
BC: Beta cell
DE: Definitive Endoderm
DNA-Pki: DNA-PK inhibitor V
EP: Endocrine Progenitor
GABA: Gamma-Aminobutyric acid
hBS: human Blastocyst derived Stem
hES: human Embryonic Stem
hESC: human Embryonic Stem Cell
hiPS: human induced Pluripotent Stem
HSC: Hematopoietic Stem Cell
iPS: Induced Pluripotent Stem
iPSC: Induced Pluripotent Stem Cell
KOSR: KnockOut™ Serum Replacement
PE: Pancreatic Endoderm
Rocki: Rho Kinase Inhibitor
SC: Stem Cell

### EXAMPLES

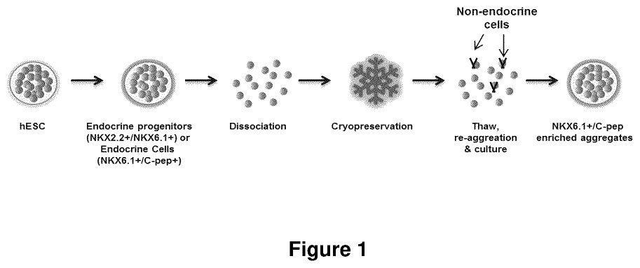

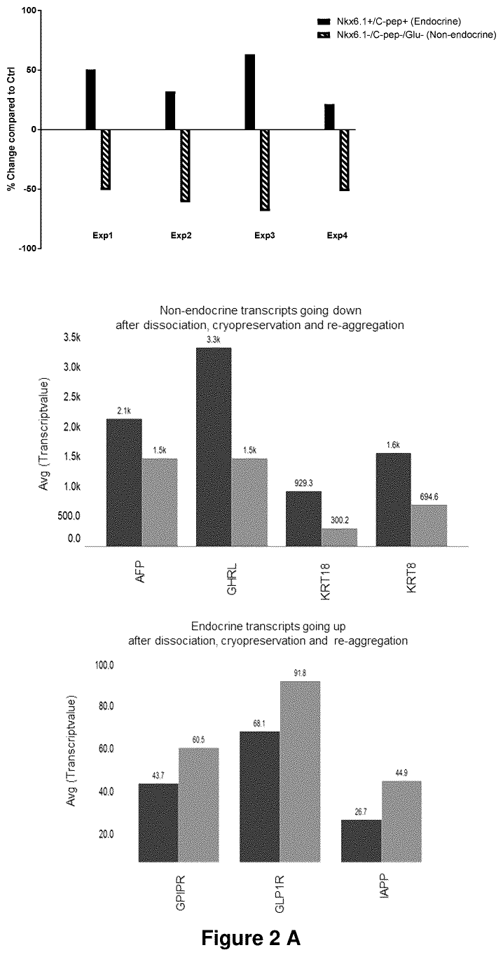

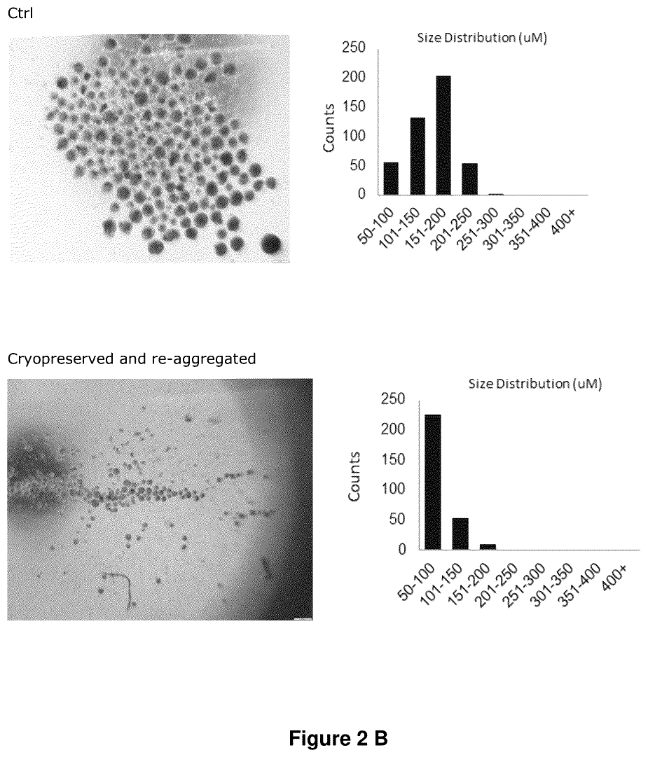

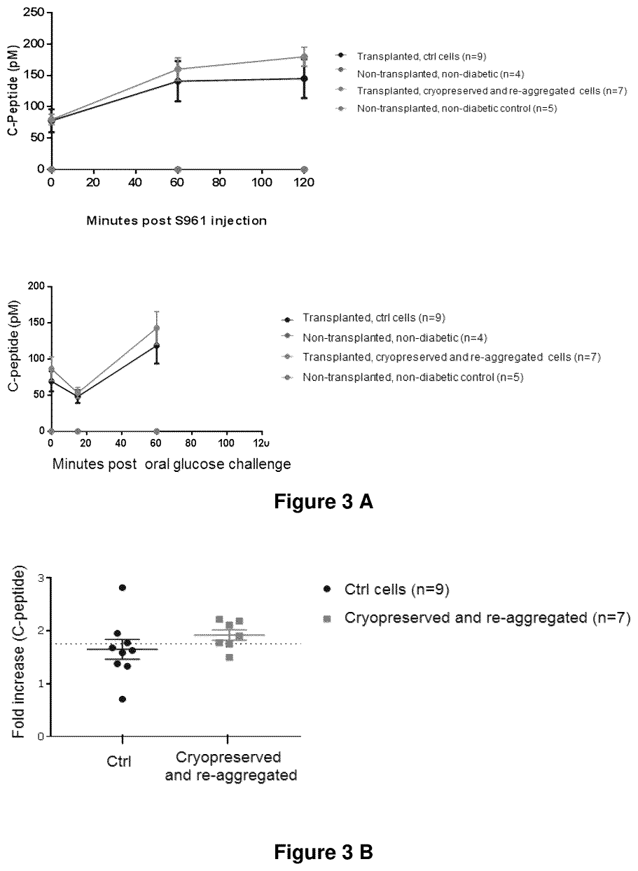

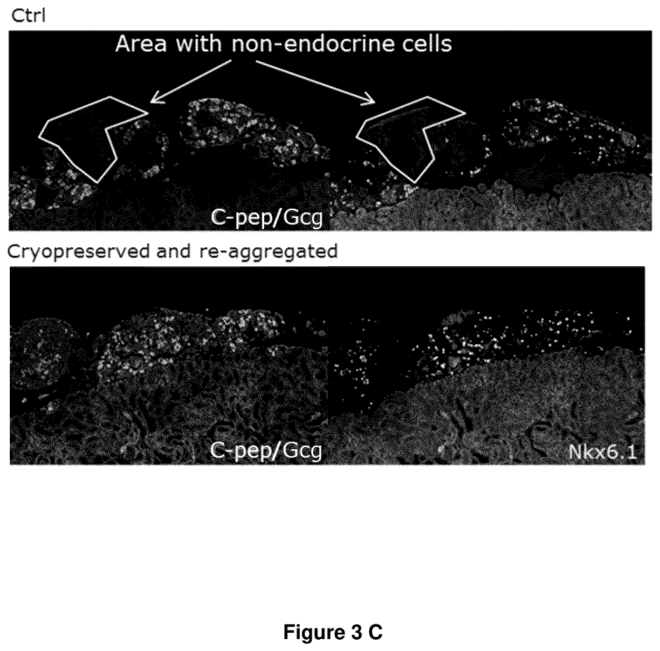

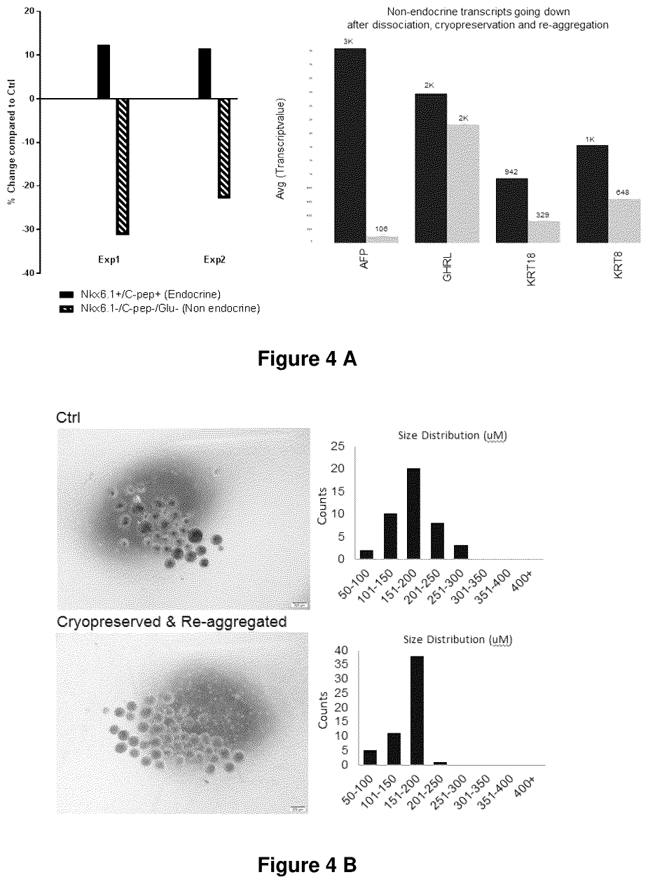

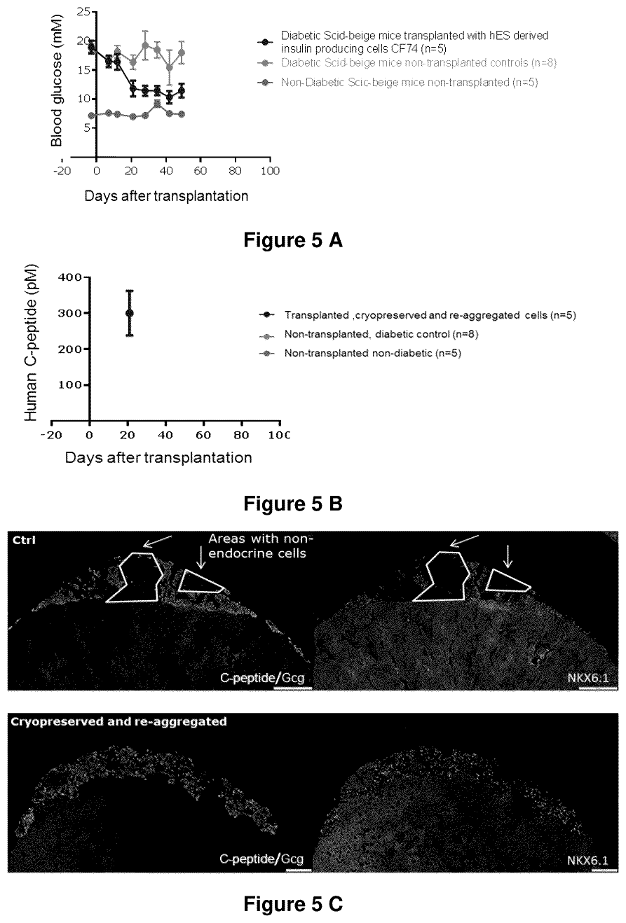

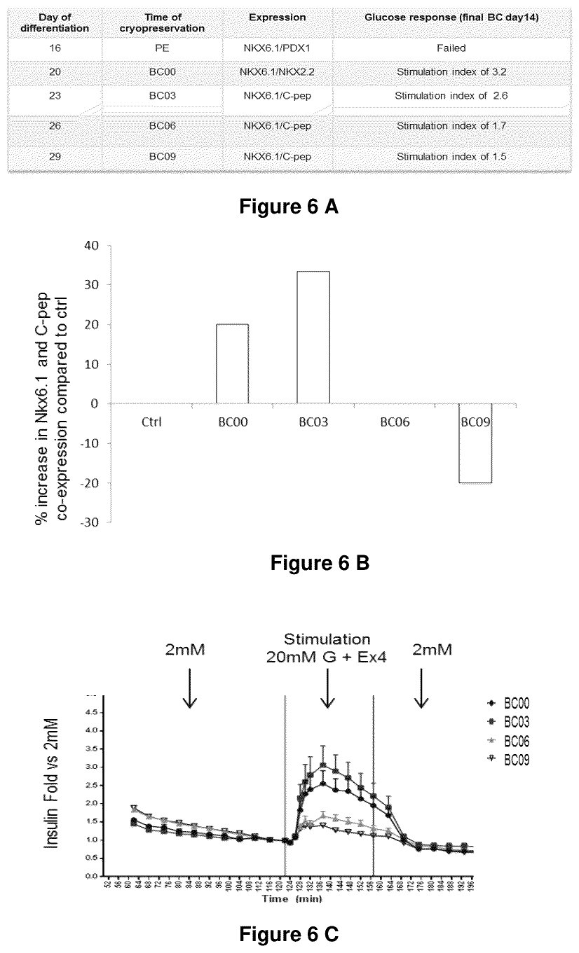

In general, the process of enriching NKX6.1 and C-peptide co-expressing cells goes through various stages. An exemplary method for enrichment is outlined in FIG. 1.

### Example 1: Preparation of Endocrine Cell Population

Protocols for obtaining endocrine progenitor cells and glucose responsive insulin secreting cells have been provided in patent applications WO2015/028614 and WO2017/144695 respectively.

### Example 2: Enrichment of NKX6.1 and C-Peptide Co-Expressing Cell Aggregates by Cryopreserving the Endocrine Progenitor Cells Co-Expressing NKX2.2 and NKX6.1

NKX2.2 and NKX6.1 co-expressing cell aggregates that have been obtained in vitro from stem cells are subjected to the following steps:

### (i) Dissociation

NKX2.2 and NKX6.1 co-expressing cell aggregates obtained from stem cells are dissociated into single cells using Accutase (Stem cell #07920). Digestion is stopped by addition of RPMI1640 medium (Gibco #61870-044) supplemented with 12% KOSR (Gibco #10828-0280) and the suspension is filtered through a 40 μm filter to remove any residual clusters.

### (ii) Cryopreservation

After centrifugation NKX2.2 and NKX6.1 co-expressing cells are re-suspended in cryopreservation media and preserved by a sequential lowering of temperature to −80° C.

### (ii) Thawing Cryopreserved Single Cells

To bring the cells back in culture, NKX2.2 and NKX6.1 co-expressing cells are quickly brought to 37° C. and washed once in pre-warmed RPMI1640 medium (Gibco #61870-044) supplemented with 12% KOSR (Gibco #10828-0280). After counting the cells are re-suspended in stage specific medium supplemented with 50 g/mL DNaseI (Sigma #11284932001) and 10 UM Rocki (Sigma #Y27632-Y0503).

(iii) Re-Aggregating the Cells Obtained after Thawing

NKX2.2 and NKX6.1 co-expressing cells are obtained after thawing are re-aggregated in Erlenmeyer flasks in a reduced volume with a density of 0.5-2 mio viable cells/mL. Re-aggregation is performed at 37° C. with horizontal shaking at 70 rpm for two days and is followed by a media change.

Endocrine Progenitor medium: RPMI1640 medium (Gibco #61870-044) supplemented with 12% KOSR (Gibco #10828-0280), 0.1% P/S (Gibco #15140-122), 10 mM Nicotinamide (Sigma #N0636), 10 UM Alk5i II (Enzo #ALX-270-445), 1 μM DZNEP (Tocris #4703), 10 μg/mL Heparin (Applichem #A3004,0250), 2.5 UM DAPT (Calbiochem #565784) and 1 μM T3 (Sigma #T6397).

After cryopreservation, endocrine progenitor cells co-expressing NKX2.2 and NKX6.1 with viability above 60% are recovered. After re-aggregation and differentiation into endocrine cells co-expressing NKX6.1 and C-peptide, said endocrine cells form clusters resulting in small and more homogeneous aggregates which may contribute to more homogeneous grafts in vivo (size ˜100 μm, <50% reduction of NKX6.1/C-PEP/GLU negative cells, increase of >50% NKX6.1 positive cells). Effects on endocrine progenitor cells co-expressing NKX2.2 and NKX6.1 phenotype in vitro are provided in FIGS. 2A and 2B respectively.

After transplantation into non-diabetic mice the dissociated, cryopreserved and re-aggregated endocrine progenitors cells co-expressing NKX2.2 and NKX6.1 are functional and secrete human C-peptide when challenged with glucose or acute insulin resistance induced by S961 (FIG. 3A). Animals receiving cells generated using a protocol with or without a dissociation, cryopreservation and re-aggregation steps have shown an increase in C-peptide during the S961 challenge. This results show that the efficacy was improved using the protocol with dissociation, cryopreservation and re-aggregation and the variation between the animals was reduced (FIG. 3B).

Immunohistochemistry analysis on kidney grafts showed that dissociation, cryopreservation and re-aggregation steps leads to an enrichment of endocrine cells types (insulin, glucagon, NKX6.1) and a reduction of non-endocrine cells types (FIG. 3C). This data might also explain the reduction of non-responders two weeks after transplantation.

### Example 3: Enrichment of NKX6.1 and C-Peptide Co-Expressing Cell Aggregates by Cryopreserving the Cells Co-Expressing NKX6.1 and C-Peptide

NKX6.1 and C-peptide co-expressing cell aggregates that have been obtained in vitro from stem cells are subjected to the following steps:

### (i) Dissociation

NKX6.1 and C-peptide co-expressing cell aggregates obtained from stem cells are dissociated into single cells using Accutase (Stem cell #07920). Digestion is stopped by addition of RPMI1640 medium (Gibco #61870-044) supplemented with 12% KOSR (Gibco #10828-0280) and the suspension is filtered through a 40 μm filter to remove any residual clusters.

### (ii) Cryopreservation

After centrifugation NKX6.1 and C-peptide co-expressing cells are re-suspended in cryopreservation media and preserved by a sequential lowering of temperature to −80° C.

(iii) Thawing Cryopreserved Single Cells

To bring the cells back in culture, NKX6.1 and C-peptide co-expressing cells are quickly brought to 37° C. and washed once in pre-warmed RPMI1640 medium (Gibco #61870-044) supplemented with 12% KOSR (Gibco #10828-0280). After counting the cells are re-suspended in stage specific medium supplemented with 50 μg/mL DNaseI (Sigma #11284932001) and 10 μM Rocki (Sigma #Y27632-Y0503).

(iv) Re-Aggregating the Cells Obtained after Thawing

NKX6.1 and C-peptide co-expressing cells obtained after thawing are re-aggregated in Erlenmeyer flasks in a reduced volume with a density of 0.5-2 mio viable cells/ml. Re-aggregation is performed at 37° C. with horizontal shaking at 70 rpm for two days and is followed by a media change.

Medium: RPMI1640 medium (Gibco #61870-044) supplemented with 12% KOSR (Gibco #10828-0280), 0.1% P/S (Gibco #15140-122), 50 μM GABA (TOCRIS #0344), 10 μM Alk5i II (Enzo #ALX-270-445), 1 μM DZNEP (Tocris #4703) and 1 μM T3 (Sigma #T6397).

After cryopreservation NKX6.1 and C-peptide co-expressing cells with viability above 90% are recovered. Upon re-aggregation of NKX6.1 and C-peptide co-expressing cells the glucose responsive insulin secreting phenotype is improved (size ˜150 μm, <25% reduction of NKX6.1/C-PEP/GLU negative cells, increase of >25% NKX6.1 positive cells) (FIGS. 4A and 4B).

In vivo, the dissociated, cryopreserved and re-aggregated endocrine cells co-expressing NKX6.1 and C-peptide have shown to efficiently lowered blood glucose which correlated with high human C-peptide secretion (FIGS. 5A and 5B).

### Example 4: Gene Expression Profile Following Cryopreservation of NKX6.1 and NKX2.2 Co-Expressing Cell Aggregates or NKX6.1 and C-Peptide Co-Expressing Cell Aggregates

Dissociation, cryopreservation and re-aggregation of cells were cryopreserved at different time-points during cell differentiation. Cells were cryopreserved at Pancreatic endoderm stage (PE), 1 day before the beginning of C-peptide expression (BC00), 2 days after the beginning of C-peptide expression (BC03), 5 days after the beginning of C-peptide expression (BC06) and 8 days after the beginning of C-peptide expression (BC09) and were all from the same batch of cells. Cells were thawed and differentiated and tested for functionality at 13 days after the beginning of C-peptide expression (BC14) in the same setup. Results shows that the glucose response and NKX6.1 and C-peptide expression are higher when cells are cryopreserved at stage BC00 and BC03 (FIG. 6A).

Expression of NKX6.1 and C-peptide was measured at BC14 using flow cytometry. Data is expressed at % compared to cells from the same batch using a protocol without a dissociation, cryopreservation and re-aggregation step. Results show that enrichment of NKX6.1 and C-peptide cells is the most efficient for cells cryopreserved at BC00 and BC03 (FIG. 6B).

At the end of experiment functionality was tested using a dynamic perfusion system. All cells responded to a challenge with 20 mM glucose and exendin-4, but the highest response was observed when cells were cryopreserved at BC00 and BC03, respectively 1 day prior to and 2 days after initiation of C-peptide expression (FIG. 6C).

1. A method of cryopreserving pancreatic endocrine cell aggregates derived in vitro from stem cells comprising the following steps:
(i) dissociating said pancreatic endocrine cell aggregates into single cells;
(ii) treating said single cells with cryopreservation medium and lowering temperature to obtain cryopreserved single cells.

2. The method of cryopreserving pancreatic endocrine cell aggregates according to claim 1, wherein said endocrine cells are endocrine cells co-expressing NKX6.1 and C-peptide or endocrine progenitor cells co-expressing NKX2.2 and NKX6.1.

3. A method of enriching endocrine cell aggregates derived in vitro from stem cells with endocrine cells co-expressing NKX6.1 and C-peptide or with endocrine progenitor cells co-expressing NKX2.2 and NKX6.1 said method comprising the following steps:
(i) dissociating said endocrine cell aggregates into single cells;
(ii) treating said single cells with cryopreservation medium and lowering temperature to obtain cryopreserved endocrine cells,
(iii) thawing said cryopreserved endocrine cells; and
(iv) re-aggregating said endocrine cells obtained after thawing.

4. The method of enriching endocrine cell aggregates according to claim 3, wherein said endocrine cells of step (i) are endocrine cells co-expressing NKX6.1 and C-peptide or endocrine progenitor cells co-expressing NKX2.2 and NKX6.1.

5. The method of enriching endocrine cell aggregates according to claim 4, wherein when said endocrine cells of step (i) are endocrine progenitor cells co-expressing NKX2.2 and NKX6.1, said method further comprises a step (v) of differentiating said endocrine progenitor cells co-expressing NKX2.2 and NKX6.1 into endocrine cell aggregates co-expressing NKX6.1 and C-peptide.

6. The method according to claim 1, wherein said stem cells are embryonic stem cells.

7. Cryopreserved single endocrine cells co-expressing NKX2.2 and NKX6.1 or co-expressing NKX6.1 and C-peptide.

8. Cryopreserved single endocrine cells obtained according to the method of cryopreserving according to claim 1.

9. (canceled)

10. Re-aggregated endocrine cells comprising at least 50%, preferentially at least 60%, more preferentially at least 70%, even more preferentially at least 80% of endocrine cells co-expressing NKX6.1 and C-peptide or endocrine progenitor cells co-expressing NKX2.2 and NKX6.1.

11. Re-aggregated endocrine cells obtained according to the method of enriching endocrine cell aggregates according to claim 3.

12.-15. (canceled)

16. The method according to claim 6, wherein said embryonic stem cells are human embryonic stem cells.

17. The method according to claim 3, wherein said stem cells are embryonic stem cells.

18. The method according to claim 17, wherein said embryonic stem cells are human embryonic stem cells.

19. A method of transplanting into a subject cryopreserved single endocrine cells according to claim 7.

20. A method of treating diabetes comprising cryopreserved single endocrine cells according to claim 7.

21. A method of transplanting into a subject re-aggregated endocrine cells according to claim 10.

22. A method of treating diabetes comprising re-aggregated endocrine cells according to claim 10.
